(19) United States
(12) Patent Application Publication
(10) Pub. No.: US 2003/0034030 A1
(43) Pub. Date: Feb. 20, 2003

(54) Medical tubing securing apparatus

(76) Inventors: Christine Carlucci, Staten Island, NY (US); Gerard Carlucci, Staten Island, NY (US)

Correspondence Address: Marguerite Del Valle, Trademark and Patent Counselors of America, P.C., 19th Floor, 915 Broadway, New York, NY, 10010, US

(21) Appl. No.: 09/930,398

(22) Filed: Aug. 15, 2001

(51) Int. Cl. A62B 7/00; A62B 9/00; A61M 15/00; A61M 16/00; A62B 18/00

(52) U.S. Cl. 128/200.24

(57) ABSTRACT

This invention relates to a device designed to comfortably and efficiently maintain medical tubing in place on the head of a hospitalized patient. The device comprises a substantially circular member fabricated from an elastic material, with one or more integrated loops. The circular member engages the head of the patient, and the elastic property of the circular member serves to secure it to the head of the patient comfortably without causing constriction of blood vessels or skin irritation. The integrated loops can receive medical tubing such as that associated with continuous positive airway pressure (CPAP) delivery systems, nasal- and oral-gastric feeding tubes, pH probes, oral suction tubes, gastric secretion tubes and tubes used for intravenous drug delivery.

### BACKGROUND OF THE INVENTION

For various reasons, it sometimes becomes necessary to secure medical tubing to the head of a hospitalized patient so that the tubing can be passed into the mouth or nose of the patient. Such tubing can provide ventilation to a patient, deliver medication or anesthesia to a patient, or establish a monitoring probe on a patient.

Examples of prior attempts to provide an apparatus to secure such tubing are disclosed in U.S. Pat. No. 5,188,101 to Tumolo, U.S. Pat. No. 4,774,946 to Ackerman, U.S. Pat. No. 4,739,757 to Edwards, U.S. Pat. No. 4,018,221 to Rennie, and U.S. Pat. No. 3,161,199 to Shaw.

Prior art devices for securing medical tubing to the head are known to be prone to slippage during use. This problem is typically remedied by taping the tubing to the patient's skin. Many patients, however, develop raw and sensitive skin from contact with the adhesive of the tape or from the repeated removal or repositioning of the tape, which makes this technique less than ideal, especially for delicate or long-term patients.

Another method to prevent slippage is to tighten the device on the head. This technique, however, cannot effectively or safely be used for patients such as premature infants, who have delicate skulls that should not be subjected to undue pressure. Tightening may also result in the unwanted constriction of blood vessels. Accordingly, many delicate patients are forced to suffer the discomforts associated with medical tape because the alternative, tightening, poses unacceptable compression or constriction risks. If taping is not used, and tightening is not an option, it is an all too common problem that medical tubing on the head shifts from its proper place, impairing the function of the tubing and/or possibly causing harm to the patient.

Other drawbacks with prior art devices reside in the components, which can themselves cause discomfort or pain, or which can otherwise negatively impact on the effectiveness of the device. For example, the arcuate thick pad disclosed in U.S. Pat. No. 4,018,221 to Rennie is bulky and massive, making it uncomfortable for the patient who must, to some extent, balance this contraption on his or her forehead. In the case of the device disclosed in U.S. Pat. No. 4,774,946 to Ackerman, the pair of yokes clipping the tubing to the headband are sharp and hard, posing the danger of injury to the patient should the device slip out of place. The yokes are also capable of causing discomfort to the patient who would feel them pressing into the side of the skull, should the patient try sleeping on his or her side.

Further, many of the prior art devices are prone to inadvertent disassembly. For example, in the case of U.S. Pat. No. 5,188,101 to Tumolo, the tying strips can become loosened such that the tubing falls out of place; the multifilamentary hook and loop device disclosed in U.S. Pat. No. 4,569,348 to Hasslinger can become caught on other equipment which can cause a disengagement of the hook and loop system; the medical tubing can slip out of the open-ended yokes disclosed in U.S. Pat. No. 4,774,946 to Ackerman.

The instant invention, on the other hand, overcomes all of these drawbacks of the prior art, by utilizing a band of soft, elastic material to encircle the head. Portions of the band are gathered to form closed loops which can hold the tubing. The instant invention thus does not irritate the skin, compress the skull or blood vessels, or incorporate bulky or dangerous components which can cause discomfort or injury. In addition, by virtue of the tubing loops being closed, the tubing cannot disengage from the securing device. Further, the device of the instant invention can be used on other parts of the body, such as the limbs, to secure medical tubing without the need for an excessive amount of medical tape.

### BRIEF DESCRIPTION OF THE INVENTION

This invention relates to an apparatus designed to comfortably and efficiently maintain medical tubing in place on the head of a hospitalized patient. The apparatus comprises a substantially circular member fabricated from an elastic material, with one or more closed loops integrated therewith. The circular member engages the head of the patient, and the elastic property of the circular member serves to secure it to the head of the patient comfortably without causing constriction of blood vessels or skin irritation. Preferably, the circular member is covered with soft, non-irritating material, to maximize comfort. Also, the interior of the circular member may be lined with material to create friction-based contact with the patient's skin, to help prevent slippage of the apparatus. The integrated loops can receive medical tubing such as that associated with continuous positive airway pressure (CPAP) delivery systems, nasal- and oral-gastric feeding tubes, pH probes, oral suction tubes, gastric secretion tubes and tubes used for intravenous drug delivery.

### BRIEF DESCRIPTION OF THE DRAWINGS

FIG. 1 is a perspective view of the apparatus of the invention in use securing continuous positive airway pressure (CPAP) tubing in place on the head of a patient.

FIG. 2 is a perspective view of the apparatus.

FIG. 3 is a top view of the band which forms the apparatus prior to the formation of the medical tubing loops.

FIG. 4 is a top view of the manufactured apparatus.

### DETAILED DESCRIPTION OF THE INVENTION

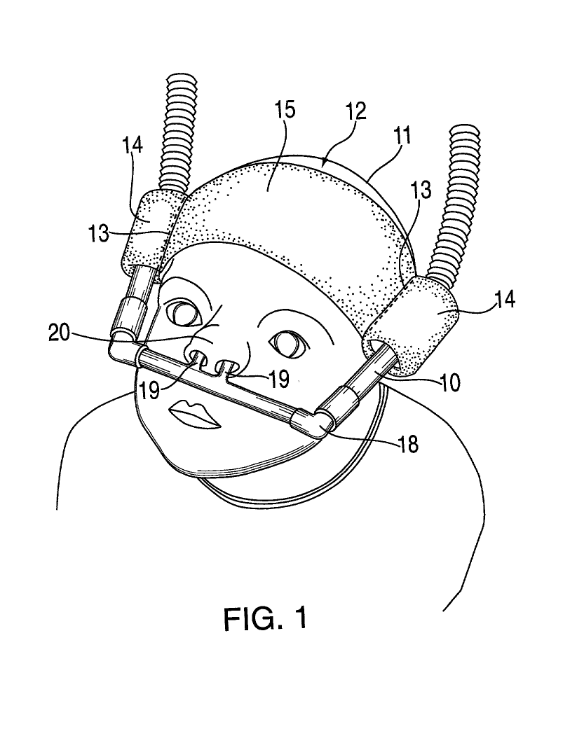

Referring to FIGS. 1, 2, 4, the device of the instant invention is denominated by numeral 12. Device 12 is made of a band of an elasticized, absorbent fabric, such as terry cloth or other suitable material and can be covered with soft, non-irritating material, to maximize comfort. The interior 17 of the band may be lined with a second material such as felt or rubberized strips to create friction-based contact with the patient's skin, to assist in preventing slippage of device 12.

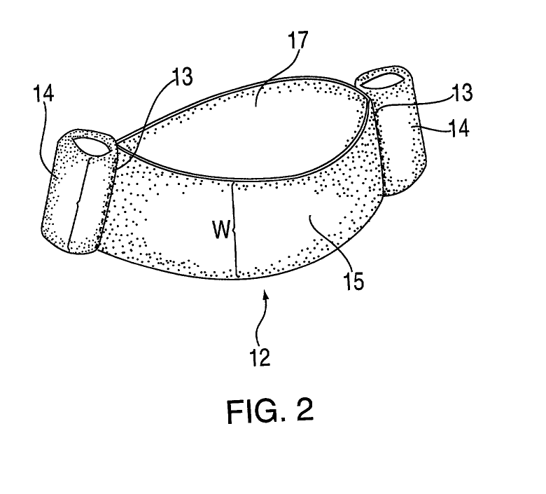

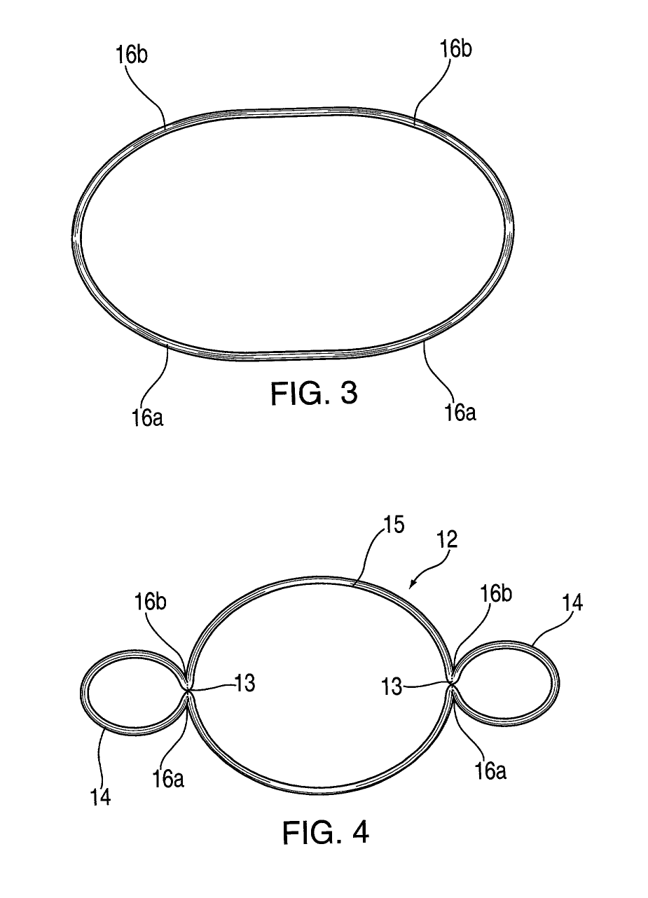

Device 12 includes a relatively large circular section 15 which will fit snugly to the head of the patient when slipped over the top of the skull. At least one loop 14 is adjacent to section 15, through which medical tubing such as that associated with a CPAP apparatus 10 can be inserted. As shown in FIGS. 2, 3 and 4, loop 14 can be formed by joining two points 16a and 16b, along the width w of the band, such as by stitching 13 or other non-disengagable fastening technique.

As shown by reference to FIG. 1, CPAP apparatus 18 is secured to the head of a patient 11 through use of the device 12. Apparatus 12 extends around the patient's head, above the ears. The tubing portions 10 of the CPAP apparatus are passed through loops 14 and the nasal cannulae 19 are positioned so as to fit into the nose 20 of the patient. There is no danger of the tubing portions pulling away from device 12, since loops 14 are closed.

Used as illustrated, device 12 is comfortably yet firmly seated on the patient's head and securely anchors medical tubing to the head, without the need for potentially injurious components. The device is of simple manufacture, and can be produced in a cost-effective manner.

What is claimed is:

1. A device to secure medical tubing to a body comprising a fabric band having at least a first closed loop and a second closed loop, wherein the first closed loop fits elastically around a portion of the body and the second closed loop is capable of receiving and holding medical tubing close to the body.

2. A device according to claim 1 wherein the portion of the body around which the first closed loop fits is a head.

3. A device according to claim 1 wherein fabric band is covered with a soft, non-irritating material.

4. A device according to claim 1 wherein the fabric band is at least partially lined with a friction creating material.

5. A device according to claim 1 wherein the fabric band is of one piece having stitching to form the closed loops.

6. A device to secure medical tubing to a body comprising a fabric band having a first closed loop, a second closed loop and a third closed loop, wherein the first closed loop fits elastically around a portion of the body and the second and third closed loops are capable of receiving and holding medical tubing close to the body.

7. A device according to claim 6 wherein the portion of the body around which the first closed loop fits is a head.

8. A device according to claim 6 wherein fabric band is covered with a soft, non-irritating material.

9. A device according to claim 6 wherein the fabric band is at least partially lined with a friction creating material.

10. A device according to claim 6 wherein the fabric band is of one piece having stitching to form the closed loops.

11. A device to secure medical tubing to a body comprising a one piece fabric band having a width wherein stitching along the width joins portions of the band to form a first closed loop, a second closed loop and a third closed loop, and wherein the first closed loop fits elastically around a portion of the body and the second and third closed loops are capable of receiving and holding medical tubing close to the body.

12. A device according to claim 11 wherein the portion of the body around which the first closed loop fits is a head.

13. A device according to claim 11 wherein fabric band is covered with a soft, non-irritating material.

14. A device according to claim 11 wherein the fabric band is at least partially lined with a friction creating material.
